(19) United States
(12) Patent Application Publication
(10) Pub. No.: US 2022/0211968 A1
(43) Pub. Date: Jul. 7, 2022

(54) TRANSPORTABLE LUNG VENTILATOR

(71) Applicant: MAGNAMED TECNOLOGIA MEDICA S/A, Paraíso (BR)

(72) Inventors: Tatsuo SUZUKI, São Paulo (BR); Toru MIYAGI KINJO, São Paulo (BR); Wataru UEDA, São Paulo (BR)

(21) Appl. No.: 17/438,635

(22) PCT Filed: Mar. 13, 2019
(86) PCT No.: PCT/BR2019/050082

§ 371 Date: Sep. 13, 2021

(51) Int. Cl. A61M 16/12; A61M 16/20; A61M 16/00

(52) U.S. Cl. CPC A61M 16/127; A61M 16/203; A61M 2016/1025; A61M 16/0012; A61M 16/0063

(57) ABSTRACT

A transportable lung ventilator allowing for automatic selection between oxygen and air for feeding a venturi type ventilation system comprising a monobloc body with at least one medical oxygen inlet; at least one compressed air inlet; and at least one ambient air inlet; at least a selector valve integrated with the monobloc body and adapted to allow, selectively, the passage of medical oxygen or compressed air; a venturi system, internal to the monobloc body, comprising an injector nozzle and a venturi tube, the injector nozzle receiving the medical oxygen or the compressed air passing through the selector valve and generating a jet towards the venturi tube, the jet dragging ambient air through the ambient air inlet to generate a mixture of gases to be ventilated to a patient; a fraction of inspired oxygen sensor that measures the concentration of oxygen in the gas mixture; and an electronic control system that controls the selector valve to allow the passage of medical oxygen or compressed air based on the oxygen concentration measured by the fraction of inspired oxygen sensor.

### FIELD OF THE INVENTION

The present invention relates to a transport pulmonary ventilator and, more specifically, to a pulmonary ventilator that allows automatic selection between oxygen and air as the motive gas of a venturi type ventilation system.

### BACKGROUND OF THE INVENTION

Different types of pulmonary ventilators for ventilation of patients with respiratory failure are known in the state of the art. Transport ventilators are non-stationary ventilators, which can be used for inter or intra-hospital transport of the patient. Thus, this type of ventilator tends to be smaller and have a more resistant body, being able to be used in transport means such ambulances or aircrafts, or even transported next to stretchers or attending devices in rescues.

The development of transport ventilators presents a technical challenge. On the one hand, the ventilators should be robust enough to handle vibrations from transport, on the other hand, should include the greatest amount of resources possible to fully meet the needs of the patient.

The differences between patients' respiratory needs are even more evident when considering the needs of adult patients and neonatal patients.

Among the variables associated with ventilation support is the fraction of inspired oxygen, also known as FiO₂, which represents the concentration of oxygen that is offered to the patient.

Conventional transport ventilators use venturi system to drag ambient air, with compressed oxygen being used as the motive gas. Thus, in this type of ventilator, the venturi system causes the oxygen under pressured to generate a vacuum that allows the dragging of ambient air in order to generate the gas mixture used in pulmonary ventilation.

These known solutions, however, have a deficiency of yield: when the oxygen flow is low, the dragging of ambient air is low; meaning, the air is not sufficiently sucked in. This low efficiency of air suction for low flow rates causes the FiO₂ to rise in the mixture.

Excess oxygen can cause damage, especially in neonates and mainly in preterms. The high concentration of oxygen in the blood, for example, can lead to abnormal retinal vessel growth (retinopathy of prematurity) and the prolonged exposure to high oxygen concentrations can lead to bronchopulmonary dysplasia, pulmonary atelectasis, and other problems.

Thus, for these patients, ventilation with air without the addition of oxygen (21% O₂) is desired. It occurs that even in ventilators that use optimized venturi systems to obtain the maximum yield in the low flow range used in neonates and preterms, the construction of the system keeps from reaching a percentage of 21% of oxygen.

A known solution of the state of the art for the high FiO₂ problem is the use of a pneumatic air/O₂ blender. This type of blender, which is coupled to the oxygen inlet, allows gas adjustment from 21% to 100%.

However, this known solution also has drawbacks, since the cost of the air/O₂ blender is very high, the device volume is significant, and the use of the pneumatic blender requires the use of two gas cylinders, the compressed air cylinder and the compressed oxygen cylinder, causing transport difficulties. In addition to the high cost and difficulty generated by the cylinders, the pneumatic blender requires the manual adjustment of FiO₂, which is uncomfortable and causes operational risks.

Additionally, even if the compressed air cylinder could be replaced by an air compressor associated with the pneumatic blender, the weight of a compressor is too large to be transported easily. Besides that, the use of the compressor in the ventilator requires the use of a reservoir to supply the demand peaks of the air flow and a dehumidifier due to the generation of water by natural humidity present in the ambient air that condenses when compressed.

Another known solution involves the use of internal turbines. In this solution, the ventilator uses an internal turbine that generates airflow with enough pressure to ventilate the lung. Although the use of a turbine with the addition of pure oxygen through a proportional valve in the gas that goes to the patient allows the adjustment of the oxygen concentration from 21% to 100%, it is a high cost solution, which requires a greater consumption and results in a ventilator with greater weight and size. In addition, the turbine generates noise and engine heat, which, if not treated properly, can lead to overheating of the air going to the patient.

Thus, there remains in the state of the art the need for a transport ventilator that is capable of meeting the needs of both adult and neonatal patients, allowing adjustment of oxygen concentration so that the fraction of inspired oxygen is suitably reached in each situation.

### Objectives of the Invention

It is an objective of the present invention to provide a compact and efficient transport ventilator, which is capable of meeting the needs of both adult and neonatal patients.

Another objective of the present invention is to provide a transport ventilator capable of adjusting oxygen concentration so that the fraction of inspired oxygen is suitably reached in each situation.

It is another objective of the present invention to provide a transport ventilator which allows an automatic selection between oxygen and air as the motive gas for a venturi type ventilation system.

Another objective of the present invention is to provide a transport ventilator adapted to utilize a small motive air source coupled to the ventilator body.

It is yet another objective of the present invention to provide a transport ventilator which dispenses the use of internal reservoir of air and dryer even when air compressor is used.

### BRIEF DESCRIPTION OF THE INVENTION

The present invention achieves these and other objectives through a transport ventilator characterized by comprising:

a monobloc body with at least one medical oxygen inlet; at least one compressed air inlet, and at least one ambient air inlet;

at least one selector valve integrated to the monobloc body and adapted to selectively allow the passage of medical oxygen or compressed air;

a venturi system, internal to the monobloc body, which comprises an injector nozzle and a venturi tube, the injector nozzle receiving the medical oxygen or compressed air that passes through the selector valve and generating a jet towards the venturi tube, the jet dragging ambient air through the inlet of ambient air to generate a mixture of gases to be ventilated to a patient;

a sensor of fraction of inspired oxygen that measures the concentration of oxygen in the gas mixture; and

an electronic control system that controls the selector valve to allow the passage of medical oxygen or compressed air based on the oxygen concentration measured by the sensor of fraction of inspired oxygen.

With this construction, the transport ventilator of the present invention allows the oxygen concentration in the inspired gas to be adjusted to values between 21% and 100% by the electronic control of the selector valve.

Preferably, the inlet of compressed air is supplied by a small compressed air source coupled to the monobloc body. The small compressed air source may be, for example, a mini compressor coupled to the monobloc body or a small air cylinder coupled to the monobloc body.

In the preferred embodiment of the present invention, the medical oxygen inlet is an oxygen pressure regulating valve, the compressed air inlet is an air pressure regulating valve, and the ambient air inlet is a one-way valve associated with a filter.

The transport ventilator further comprises a proportional valve which receives the oxygen or compressed air that passes through the selector valve and directs it to the injector nozzle; a proportional oxygen valve that allows the passage of medical oxygen from the oxygen pressure regulating valve directly to the gas mixture to be ventilated; and a proportional air valve that allows the passage of compressed air from the air pressure regulating valve directly to the gas mixture to be ventilated.

Thus, the electronic control system can open and close the proportional oxygen valve and proportional air valve based on the measurement of the output flow of gas mixture measured by a flow sensor, so that it is possible to complete the current volume required for the adjusted FiO₂ in the ventilator.

### BRIEF DESCRIPTION OF DRAWINGS

The present invention will be described in more detail below along with references to the attached drawings, in which:

FIG. 1—is a perspective view of the transport ventilator according to the preferred embodiment of the present invention;

FIG. 2—is a second perspective view of the transport ventilator according to the preferred embodiment of the present invention;

FIG. 3—is a cross-sectional view of the transport ventilator according to the preferred embodiment of the present invention; and

FIG. 4—is a schematic illustration of the integrated electronic blender comprised by the transport ventilator according to the preferred embodiment of the present invention.

### DETAILED DESCRIPTION OF THE INVENTION

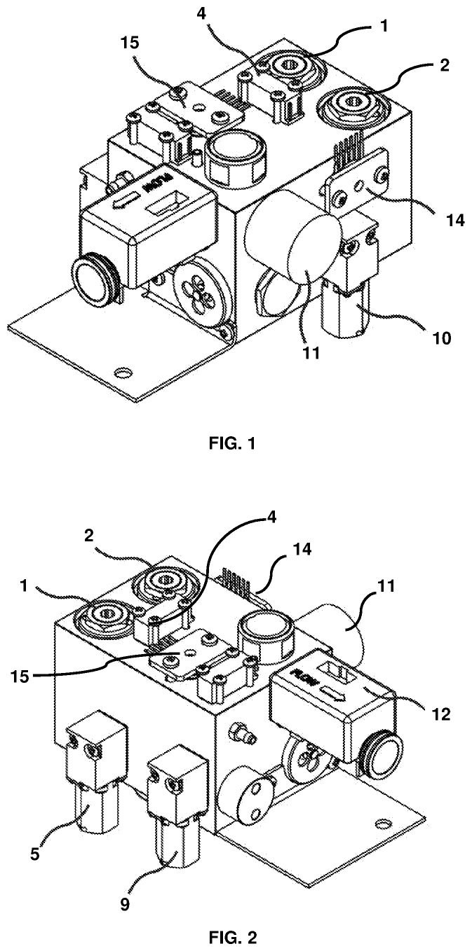

The present invention will be further described based on an example of preferred embodiment of the transport ventilator according to the present invention illustrated in FIGS. 1 to 4.

FIG. 1 shows a first perspective view of the transport ventilator of the present invention.

The ventilator comprises a monobloc body structure where the ventilator components are inserted or attached and within which the gas passageways are formed.

FIG. 1 shows the outside part of the monobloc where an oxygen pressure regulating valve 1 is fixed, through which pressurized medical oxygen enters, and an air pressure regulating valve 2, through which the compressed air enters.

The air pressure regulating valve 2 may be associated with a one-way valve 3, which is schematically illustrated in FIG. 4. The one-way valve 3 prevents the return of the gas in the absence of the air source.

The compressed air can be any suitable source, such as a hospital compressed air supply network, a compressed air cylinder with a regulating valve or an air compressor.

The oxygen source can be any suitable source, such as a hospital oxygen supply network or an oxygen cylinder with a regulating valve.

As illustrated in FIGS. 1 and 2, the ventilator can also comprise, on the outer face of the monobloc body, an air pressure measuring point 14 and an oxygen pressure measuring point 15.

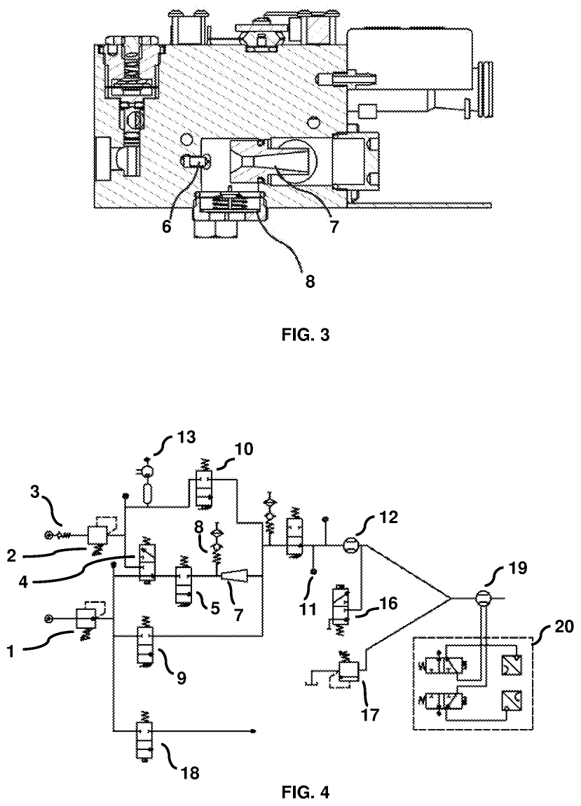

A venturi system, which comprises an injector nozzle 6 and a venturi tube, uses the pressure of the entering gas to generate a vacuum which allows the dragging of ambient air. As shown in FIG. 3, ambient air enters through an ambient air inlet assembly formed by a one-way valve 8 associated with a filter. As shown in the cross-sectional view of FIG. 3, the injector nozzle 6 and the venturi system 7 are formed inside the monobloc body.

A solenoid valve 4 is provided to act as a gas selector valve for the mixture to be delivered to the patient. Thus, the solenoid valve 4 allows alternative passage of air or oxygen. As it will be explained later, the selector solenoid valve 4 allows automatic adjustment of the oxygen concentration in FiO₂.

In the preferred embodiment of the invention, the ventilator also comprises at least one proportional valve 5, preferably fixed to the face of the monobloc body. The proportional valve 5, shown in FIGS. 2 and 4, is a pneumatic valve commanded by an electrical signal whose flow is proportional to the fed electric current.

Thus, the gas (either oxygen or air), exits the respective regulating valve 1 or 2 with the set pressure, passes through the solenoid valve 4 and continues to the proportional valve 5.

The proportional valve 5 provides the gas to the injector nozzle 6 of the venturi system 7 (see FIGS. 3 and 4). Thereby, the gas, upon passing through the injector nozzle 6, becomes a gas jet dragging ambient air which enters the monobloc through the ambient air inlet assembly. The gas mixture that exits the venturi system 7 goes to the patient.

The transport ventilator of the present invention allows the oxygen concentration in the inspired gas to be adjusted to values between 21% and 100% by the electronic control of the selector solenoid valve 4.

When the selector solenoid valve 4 is open for only the oxygen inlet (i.e., when the solenoid valve 4 is “off”), the oxygen is directed to the injector nozzle 6 dragging the ambient air through the ambient air inlet assembly. In this situation, the gas mixture formed in the venturi tube has a minimum FiO₂ of about 35% O₂ in a higher yield flow rate (i.e., the minimum oxygen concentration achieved in the gas mixture in the venturi when the inlet gas is oxygen is at least about 35%, since the O₂ dragged is mixed with ambient air which has about 21% of oxygen). This minimum value tends to increase when there is a higher pressure at the exit of the venturi tube 7 or when a very low flow rate is set as that suitable for neonates (i.e.; when less ambient air is absorbed).

Thus, in order to achieve an O₂ concentration of less than 35% in FiO₂, the transport ventilator of the present invention allows the solenoid valve 4 to be selectively “activated” to allow the use of the compressed gas as a motive gas (i.e., the selector valve 4 starts to allow the passage of compressed air). In this situation, the O₂ that enters into the mixture is that one from ambient air which was dragged by the ambient air inlet assembly. Thus, in this situation, the FiO₂ will be 21% of O₂ from very low flow to the maximum air flow that can be inhaled.

This control of the selector solenoid valve is automatically performed by a software-based control system based on the oxygen concentration measured by an fraction of inspired oxygen sensor 11. Thus, the solenoid valve 4 allows to choose, selectively, between the passage of medical O₂ and the passage of compressed air until the measured oxygen concentration corresponds to the concentration of O₂ in the FiO₂ desired for the situation of use.

In a preferred embodiment of the ventilator of the present invention, the transport ventilator further includes a proportional oxygen valve 9 that allows the passage of oxygen directly, without passing through the injector nozzle. Preferably, the proportional valve 9 is a high flow valve.

Similarly, in the preferred embodiment, the transport ventilator includes a proportional air valve 10 that allows the passage of air directly, without passing through the injector nozzle. Preferably, the proportional valve 10 is a high flow valve.

The proportional oxygen 9 and air 10 valves have the objective of completing the current volume required for the FiO₂ adjusted in the ventilator. This control is done by a control software, which, based on the information received from the flow sensor 12, adjusts the current volume based on the measurement of the flow exiting the monobloc body.

Thus, the control software adjusts the current volume by opening and closing the proportional oxygen valves 9 and air 10.

It should be noted that the algorithm of the control software prioritizes the use of the venturi system, so that the proportional oxygen 9 and air 10 are only opened to complete the required current volume for FiO₂.

Thus, in a situation where the selector solenoid valve 4 only allows the passage of air as motive gas and where, at the maximum flow provided by the venturi, the ventilator cannot ventilate with the required volume, the proportional air valve 10 and/or the proportional O₂ valve 9 open to complete the adjusted volume in the FiO₂ required.

In the preferred embodiment of the present invention, the transport ventilator is also adapted to be coupled thereto by a mini compressor or small air cylinder 13 as a source of the compressed air used as motive gas, as shown in the diagram of FIG. 4. The possibility of coupling the small air source, associated with the selector valve 4 and the venturi system, allows the transport ventilator to provide good autonomy, since air consumption by the venturi system is low.

It should be noted that the compressor or small cylinder could also be inserted into the monobloc body itself, increasing even further the compaction of the assembly.

In the illustrative example of FIG. 4, the pneumatic scheme of the transport ventilator further comprises:

a safety valve 16, which opens in case of overpressure and reliefs the pressure of the flow delivered to the patient, thereby avoiding barotrauma;

an expiratory valve 17, which closes, following an electric command, to direct the gas to the lung of a patient during the inspiration/inhalation phase and opens to exhale the gas from the lung during the expiration/exhalation phase;

a solenoid valve 18 which may be used to allow the gas flow to be used by an accessory device such as a nebulizer or may be connected to a tube to deliver a gas jet directly to the tip of an intubation cannula (knows as—TGI/tracheal gas injection) to aid in expelling the CO₂ that sometimes accumulates in the lung;

a proximal flow sensor 19 which, when activated, monitors the gases than enter and leave the mouth of the patient; and

an offset arrangement 20, comprising two three-way solenoid valves, intended to zero the offset of the pressure differential transducers that measure the inspiratory and expiratory flows.

Therefore, having described examples of embodiments of the present invention, it should be understood that the scope of the present invention covers other possible variations of the inventive concept described, being limited only by the content of the claims, including possible equivalents.

1. Transport A transportable lung ventilator characterized by comprising:
a monobloc body with at least one medical oxygen inlet; at least one compressed air inlet, and at least one ambient air inlet;
at least one selector valve integrated to the monobloc body and adapted to allow, selectively, the passage of medical oxygen or compressed air;
a venturi system, internal to the monobloc body, that includes an injector nozzle and a venturi tube, the injector nozzle receiving the medical oxygen or the compressed air passing through the selector valve and generating a jet towards the venturi tube, the jet dragging ambient air through the ambient air inlet to generate a mixture of gases to be ventilated to a patient;
a fraction of inspired oxygen sensor that measures the concentration of oxygen in the gas mixture; and
an electronic control system that controls the selector valve to allow the passage of medical oxygen or compressed air based on the oxygen concentration measured by the fraction of inspired oxygen sensor.

2. The ventilator, according to claim 1, wherein the selector valve is a solenoid valve.

3. The ventilator, according to claim 2, wherein the compressed air inlet is fed by a small compressed air source coupled to the monobloc body.

4. The ventilator, according to claim 3, wherein the small compressed air source is selected from a mini compressor coupled to the monobloc body or a small cylinder coupled to the monobloc body.

5. The ventilator, according to claim 1, wherein the medical oxygen inlet is an oxygen pressure regulating valve, the compressed air inlet is an air pressure regulating valve, and the ambient air inlet is a one-way valve associated with a filter.

6. The ventilator, according to claim 5, further comprising a proportional valve that receives the oxygen or compressed air which passes through the selector valve and forwards it to the injector nozzle.

7. The ventilator, according to claim 6, further comprising:
an oxygen proportional valve that allows the passage of medical oxygen from the oxygen pressure regulating valve directly into the gas mixture to be ventilated; and
an air proportional valve that allows the passage of compressed air from the air pressure regulating valve directly into the gas mixture to be ventilated;
wherein the electronic control system opens and closes the oxygen proportional valve and air proportional valve based on the measurement of the exit flow of the gas mixture measured by a flow sensor.
